(19) United States
(12) Patent Application Publication
(10) Pub. No.: US 2011/0223202 A1
(43) Pub. Date: Sep. 15, 2011

(54) CATIONIC CORE-SHELL PEPTIDE NONOPARTICLES

(76) Inventors: Yi-Yan Yang, Singapore (SG); Lihong Liu, Singapore (SG)

(21) Appl. No.: 12/809,009

(22) PCT Filed: Dec. 18, 2008
(86) PCT No.: PCT/SG08/00492

§ 371 Date: Mar. 3, 2011

Related U.S. Application Data
(60) Provisional application No. 61/006,089, filed on Dec. 18, 2007.

(51) Int. Cl. A61K 38/16; C07K 14/00; C07K 7/06; C07K 5/00; C07K 7/08; A61K 9/10; A61P 31/00; A61P 31/04; B82Y 5/00

(52) U.S. Cl. 424/400; 530/300; 530/327; 530/331; 530/330; 530/329; 530/328; 530/326; 530/325; 530/324; 514/1.1; 514/2.4; 514/3.3; 514/3.5; 514/2.3; 977/773

(57) ABSTRACT

The invention discloses an amphiphilic antimicrobial substance comprising a hydrophobic portion coupled to a cationic oligopeptide portion. The cationic oligopeptide portion may comprise a protein transduction domain coupled to a cationic oligopeptide group.

### TECHNICAL FIELD

The present invention relates to cationic core-shell peptide nanoparticles, their formation and use as antimicrobial agents.

### BACKGROUND OF THE INVENTION

Brain inflammatory diseases such as meningitis and encephalitis are among the top ten infectious causes of death, which may be caused by different bacteria such as Bacillus anthrax and Bacillus subtilis or fungi. HIV-infected patients can easily be infected with fungi due to their damaged immune systems, and Candida albicans is the most frequently found fungus in meningitis. Satratoxin G from Stachybotrys chartarum has also been reported to cause brain inflammation. Brain infection can be severe as it may result in hearing loss, learning disability or brain damage. Despite antibiotic treatment, there is high mortality and morbidity because of the difficulty in delivering drugs across the blood-brain barrier (BBB) to the brain. Cationic antimicrobial peptides have recently received increasing attention due to their broad-spectrum activities and ability of combating multi-drug resistant microbes. There is therefore a need for a new form of cationic peptide having improved antimicrobial activity, preferably a form of cationic antimicrobial peptide that can cross the blood-brain barrier.

### OBJECT OF THE INVENTION

It is the object of the present invention to substantially at least partially satisfy the above need.

### SUMMARY OF THE INVENTION

In a first aspect of the invention there is provided an amphiphilic antimicrobial substance comprising a hydrophobic portion coupled to a cationic oligopeptide portion.

The following options may be used in conjunction with the first aspect, either individually or in any suitable combination.

The cationic oligopeptide portion may comprise arginine residues. It may comprise lysine residues. It may comprise both arginine and lysine residues. It may be between 5 and 35 peptide units in length. It may comprise a protein transduction domain. The protein transduction domain may be a terminal domain. It may be TAT (YGRKKRRQRRR). It may be coupled to a cationic oligopeptide group. Thus the cationic oligopeptide portion may comprise the protein transduction domain coupled to the cationic oligopeptide group. The cationic oligopeptide group may comprise arginine groups and/or lysine groups. It may consist of arginine groups. It may consist of lysine groups. It may consist of lysine and arginine groups. It may have from 2 to about 15 lysine and/or arginine groups. It may for example be R₆. It may be coupled to the hydrophobic portion by means of a spacer. Thus the cationic oligopeptide group may comprise the protein transduction domain coupled to the cationic oligopeptide group, which is in turn coupled to the spacer. The spacer may be relatively hydrophilic. It may be an oligopeptide group. It may be uncharged. It may be an uncharged oligopeptide group. It may be from 1 to about 10 amino acids long. It may comprise, or may consist of, glycine residues. It may for example be G₃. In the event that the spacer is an oligopeptide group, it may be linked to the hydrophobic group through its N-terminus. Additionally or alternatively the spacer may comprise an amino acid comprising a functional group, such as carboxylic acid (e.g. aspartic acid-D and glutamic acid-E), amine (e.g. lysine-K) and hydroxyl group (e.g. serine-S). In this event the spacer group may be coupled to the hydrophobic portion through said functional group.

In some embodiments the cationic oligopeptide group is R₆ and the spacer is G₃, wherein the terminal glycine residue is bonded to the hydrophobic portion through its N-terminus.

The hydrophobic portion may be a C4 to C40 group. It may comprise, or may be, a steroid group. The steroid group may be a cholesteryl group. It may comprise, or may consist of, a hydrophobic polymer. The hydrophobic polymer may be biodegradable.

In one embodiment the antimicrobial substance is CholG₃R₆TAT, wherein Chol represents a cholesteryl group and TAT represents YGRKKRRQRRR. In another embodiment the antimicrobial substance is CholG₃K₆TAT. In either of these embodiments, the antimicrobial substance may be dispersed in an aqueous matrix as micelles or nanoparticles of mean diameter less than about 700 nm.

The antimicrobial substance may be in the form of micelles or nanoparticles. The micelles or nanoparticles have a mean diameter of about 100 to about 700 nm.

The antimicrobial substance may have a minimum inhibitory concentration (MIC) against any one of, optionally each of, Bacillus subtilis, Candida albicans and Stachybotrys chartarum of less than about 15 micromolar. It may be active against one, two or all of bacteria, yeast and fungi. It may be active against both bacteria and fungi.

The antimicrobial substance may be capable of crossing the blood-brain barrier.

In a second aspect of the invention there is provided a process for making an amphiphilic antimicrobial substance according to the first aspect, said process comprising coupling a hydrophobic compound to a cationic oligopeptide.

The following options may be used in conjunction with the second aspect, either individually or in any suitable combination.

The process may comprise reacting the hydrophobic compound with the N-terminus of the cationic oligopeptide or with a functional group in said cationic oligopeptide. The cationic oligopeptide may comprise an uncharged oligopeptide spacer having a cationic oligopeptide group coupled to its C-terminus, said cationic oligopeptide group having a protein transduction domain coupled to its C-terminus.

The hydrophobic compound may be a haloformate ester.

The process may additionally comprise the step of dispersing the antimicrobial substance in water so as to form nanoparticles or micelles of the antimicrobial substance in the water. The nanoparticles or micelles may each comprise a hydrophobic core surrounded by a hydrophilic shell. In this case, the process may comprise incorporating one or more therapeutic agents into the cores of the nanoparticles or micelles.

In an embodiment there is provided a process for making an amphiphilic antimicrobial substance according to the first aspect, said process comprising coupling a steroidal chloroformate ester to the N-terminus of a cationic oligopeptide.

In another embodiment there is provided a process for making an amphiphilic antimicrobial substance according to the first aspect, said process comprising:

using a solid state method to synthesise an cationic oligopeptide, said oligopeptide comprising, an oligopeptide linker portion having a protein transduction domain coupled to the C-terminus thereof; and

coupling a steroidal chloroformate ester to the N-terminus of said cationic oligopeptide.

In another embodiment there is provided a process for making an amphiphilic antimicrobial substance according to the first aspect, said process comprising:

using a solid state method to synthesise an cationic oligopeptide, said oligopeptide comprising an oligopeptide linker portion having a protein transduction domain coupled to the C-terminus thereof; and

coupling a steroidal chloroformate ester to the N-terminus of said cationic oligopeptide to form the antimicrobial substance; and

forming micelles or nanoparticles of said antimicrobial substance in an aqueous matrix.

The invention also provides an amphiphilic antimicrobial substance made by the process of the second aspect. The amphiliphilic antimicrobial substance may be in the form of nanoparticles or micelles, each comprising a hydrophobic core surrounded by a hydrophilic shell. In this case, the cores of the nanoparticles or micelles may comprise one or more therapeutic agents.

In a third aspect of the invention there is provided a method for killing microorganisms comprising exposing said microorganisms to an antimicrobial substance according to the first aspect, or to an antimicrobial substance made by the process of the second aspect.

The microorganisms may be bacteria, yeast or fungus or may be a mixture of any two or all of these.

The concentration of the antimicrobial substance to which the microorganisms is exposed may be less than about 15 micromolar.

In some embodiments of the method of the third aspect, the microorganisms are pathogens located in a patient. In these embodiments the step of exposing may comprise administering said antimicrobial substance to the patient. The pathogens may be located in the brain of the patient. In this event the step of exposing may comprise allowing the antimicrobial substance to cross the blood-brain barrier of said patient.

In other embodiments of the method of the third aspect, the killing does not comprise administering the antimicrobial substance to the patient. It may not be a method for treatment of a condition in a patient.

In a fourth aspect of the invention there is provided use of an antimicrobial substance according to the first aspect, or made by the process of the second aspect for the manufacture of a medicament for the treatment of an infection in a subject, said antimicrobial substance being effective in treatment of said infection.

The infection may be an infection of the brain of the subject.

The antimicrobial substance may be in the form of nanoparticles or micelles, each comprising a hydrophobic core surrounded by a hydrophilic shell. In this case, the cores of the nanoparticles or micelles may comprise one or more therapeutic agents. The one or more therapeutic agents may be effective in treatment of said infection. The medicament may be suitable for delivery of the one or more therapeutic agents to the subject.

In a fifth aspect of the invention there is provided use of an antimicrobial substance according to the first aspect, or made by the process of the second aspect, in therapy.

In a sixth aspect of the invention there is provided a pharmaceutical composition comprising an antimicrobial compound according to the first aspect, or made by the process of the second aspect, together with one or more pharmaceutically acceptable carriers, diluents and/or adjuvants.

The antimicrobial compound may be in the form of nanoparticles or micelles in an aqueous matrix. The micelles or nanoparticles may comprise a hydrophilic shell surrounding a hydrophobic core. The hydrophilic shell may comprise the cationic oligopeptide portion. The hydrophobic core may comprise the hydrophobic portion. The hydrophobic core may also comprise a hydrophobic substance. The hydrophobic substance may be a therapeutic substance, for example an anticancer drug, a small molecule antibiotic or other suitable hydrophobic therapeutic substance.

Thus in an embodiment there is provided an antimicrobial compound in the form of nanoparticles or micelles of an amphiphilic antimicrobial substance, said substance comprising a hydrophobic portion coupled to a cationic oligopeptide portion, wherein a hydrophilic shell of said nanoparticles or micelles comprises the cationic oligopeptide portion and a hydrophobic core of said nanoparticles or micelles comprises the hydrophobic portion and a hydrophobic therapeutic substance.

### BRIEF DESCRIPTION OF THE DRAWINGS

A preferred embodiment of the present invention will now be described, by way of an example only, with reference to the accompanying drawings wherein:

FIG. 1 illustrates rational peptide design and images of peptide nanoparticles: a. schematic of the designed peptides with (1) cholesterol, (2) glycine, (3) arginine and (4) TAT; b and c: scanning electron micrographs of nanoparticles.

FIG. 2 shows scanning electron micrographs of Bacillus subtilis (A) and Candida albicans (B) before (A1, A2, B1, B2) and after treatment of nanoparticles of 13.0 micromolar for 30 (B3), 90 (A3, A4), 100 (B4, B5) and 200 minutes (B6) and 26.0 micromolar for 90 minutes (A5);

FIG. 3 illustrates dose-dependent of hemolytic activity of nanoparticles in comparison with amphotericin B.

FIG. 4 Transport of FITC-loaded nanoparticles across the blood-brain barrier. Hippocampus brain sections of rats at 4 hours after i. v. injection. A. FITC; B. FITC-loaded nanoparticles.

FIG. 5 shows a plot of I339/I334 ratio as a function of logarithm of peptide concentration (Log C) in DI water.

FIG. 6 illustrates growth of Bacillus subtilis. (A), Candida albicans (B) and Stachybotrys chartarum (C) as a function of incubation time. Incubation of Stachybotrys I was stopped at the logarithmic phase to avoid inaccurate O.D. readings caused by broth evaporation and formation of bulky hyphae.

FIG. 7 illustrates dose-dependent growth inhibition of Bacillus subtilis (A, MIC: 10.7 μM), Candida albicans (B, MIC: 10.8 μm) and Stachybotrys chartarum (C, MIC: 11.0 μM) in the presence of peptide nanoparticles and conventional antifungal agents. The incubation time of each microorganism was chosen based on its growth curve (in FIG. 5). Incubation was stopped once the stationary phase was reached.

FIG. 8 illustrates dose-dependent growth inhibition of Bacillus subtilis (A, MIC: 290.0 μM) and Candida albicans (B, MIC: 289.0 μM) in the presence of G3TAT.

FIG. 9 illustrates dose-dependent growth inhibition of Bacillus subtilis (A, MIC: 75.0 μM) and Candida albicans (B, MIC: 75.0 μM) in the presence of G3R6TAT.

FIG. 10 illustrates dose-dependent growth inhibition of Bacillus subtilis (A, MIC: 242.0 μM) and Candida albicans (B, MIC: 242.0 μM) in the presence of G3R12.

FIG. 11 illustrates dose-dependent growth inhibition of Bacillus subtilis (A, is MIC: >444.4 μM) and Candida albicans (B, MIC: >444.4 μM) in the presence of G3R6.

FIG. 12 illustrates dose-dependent growth inhibition of Bacillus subtilis in the presence of penicillin G (A, MIC: 1074 μM) and doxycycline (B, MIC: 13.5 μM) (16 hours of incubation).

FIG. 13 shows MALDI-TOF mass spectra of G3R6TAT and CG3R6TAT. The spectrum of G3R6TAT shows its theoretical molecular weight at 2667 Da, indicating the successful synthesis of the peptide. The theoretical molecular weight of CG3R6TAT was 3080 Da, which appears in the spectrum of CG3R6TAT, indicating successful conjugation of cholesterol.

FIG. 14 shows 1H-NMR spectra of CG3R6TAT and G3R6TAT in d-DMSO. The weak and multiple peaks at δ 0.7-1.1 (signal a) were from cholesterol. The multiple peaks at δ 6.7-8.5 (signal b) were attributed to the protons from the benzene ring in tyrosine. These findings further prove successful conjugation of cholesterol onto the peptide.

### DETAILED DESCRIPTION OF THE INVENTION

The present invention relates to cationic core/shell nanoparticles which are self-assembled from amphiphilic oligopeptide-based molecules that contain a cell-penetrating peptide. They may be used as antimicrobial agents. They may be capable of crossing the blood-brain barrier (BBB).

The invention provides an amphiphilic antimicrobial substance comprising a hydrophobic portion coupled to a cationic oligopeptide portion. It may comprise, or consist essentially of, a single hydrophobic portion coupled to a single cationic oligopeptide portion. Thus it may have structure A-B, wherein A is the hydrophobic portion and B is the cationic oligopeptide portion. The hydrophobic portion may not be repeated within the oligopeptide portion. The oligopeptide portion may comprise a spacer linking the hydrophobic portion to cationic residues in the cationic oligopeptide portion. The spacer may be relatively hydrophilic. It may comprise, or may consist of, an uncharged oligopeptide group e.g. oligoglycine. The substance may be capable of crossing the BBB. It may be active against infection microorganisms in a brain.

In the present specification, the following single letter codes for amino acids have been used: tyrosine—Y; glycine—G; arginine—R; lysine—K; glutamine—Q; histidine—H. These are in accordance with the standard single letter codes for amino acids. Any one or more may (independently) be in the naturally occurring optical isomer or in the non-naturally occurring optical isomer

The cationic oligopeptide portion may comprise arginine residues. It may comprise other amino acids capable of providing cationic character. It may comprise lysine residues. It may comprise a single type of amino acid (e.g. it may be an oligoarginine residue) or it may comprise more than one type of amino acid, e.g. 2, 3, 4, 5 or 6 types of amino acid. The amino acids may be in blocks or may not be in blocks, or some may be in blocks and some may be not in blocks. The cationic oligopeptide portion may comprise a block of cationic amino acids (optionally the same amino acids) and a block of non-ionic amino acids (optionally the same amino acid). The block of non-ionic amino acids may function as a spacer. The block of non-ionic amino acids may be coupled directly to the hydrophobic portion. The cationic oligopeptide portion may be between 5 and 35 peptide units (i.e. amino acid residues) in length, or 5 to 20, 5 to 10, 10 to 35, 20 to 35, 10 to 20 or 15 to 20, e.g. 5, 6, 7, 8, 9, 10, 11, 12, 13, 14, 15, 16, 17, 18, 19, 20, 21, 22, 23, 24, 25, 30 or 35 peptide units in length.

In certain embodiments of the invention, the cationic oligopeptide portion comprises a protein transduction domain. This domain may enable the antimicrobial substance to pass the blood-brain barrier and/or may enhance cell membrane penetration. This may be beneficial in enabling the use of the antimicrobial substance in treating brain diseases or in yielding an enhanced antimicrobial activity. The protein transduction domain may be a transduction domain from a naturally occurring protein. It may be for example the protein transduction domain from the transcriptional activator Tat protein of the human HIV-1 (human immunodeficiency virus type 1). This transduction domain is TAT (YGRKKRRQRRR). An analogue of TAT may be used in which conservative substitutions of one or more amino acids have been made. The analogue should have similar properties to TAT in regard to crossing the BBB and/or enhancing cell penetration. The protein transduction domain may be a cell penetrating domain. The protein transduction domain may be a terminal domain, i.e. it may be located at the end (terminus) of the molecules of the amphiphilic antimicrobial substance. In the case where the transduction domain is TAT, the terminal R of TAT may be located at the end of the molecules. The terminal location may render it more active in penetrating the BBB than if it were located in a non-terminal position.

The protein transduction domain, if present, may be coupled to the hydrophobic portion by a spacer. Suitable spacers include oligopeptide spacers. Commonly a cationic oligopeptide group is located between the protein transduction domain and the spacer. The cationic oligopeptide group may comprise arginine residues and/or lysine and/or histidine residues to confer cationic nature thereon. It is thought that the cationic nature of the linker group may contribute to the antimicrobial activity of the antimicrobial substance. The protein transduction domain may also contribute to the antimicrobial activity. The presence of cationic groups between the spacer and the protein transduction domain may influence the conformation of the antimicrobial substance, particularly when in the form of nanoparticles or micelles, so as to render the substance more biologically active. The length of the spacer plus the cationic oligopeptide group may be between about 5 and about 15 peptide units, or 5 to 10, 10 to 15 or 7 to 11, e.g. 5, 6, 7, 8, 9, 10, 11, 12, 13, 14 or 15 peptide units. The cationic oligopeptide group may comprise a single type of amino acid (e.g. it may be an oligoarginine residue) or it may comprise more than one type of amino acid, e.g. 2, 3, 4, 5 or 6 types of amino acid. The amino acids may be in blocks or may not be in blocks, or some may be in blocks and some may be not in blocks. It may for example comprise a diblock oligopeptide. It may comprise a diblock oligopeptide in which one, optionally both, of the amino acid units of the blocks are cationic. The cationic amino acid residues may be located towards the C terminus of the oligopeptide group. The oligopeptide group may for example comprise R₆ or H₆ or K₆. The spacer may comprise, or consist of, uncharged or non-ionic peptide residues. It may be relatively hydrophilic. It may have hydrophilicity intermediate between the hydrophobic portion and the cationic oligopeptide portion. It may be between 1 and about 6 peptide units long, or about 1 to 3, 3 to 6 or 2 to 4 peptide units long. It may be for example 1, 2, 3, 4, 5 or 6 peptide units long. It may comprise, or consist of glycine units. The N-terminal amino acid of the spacer may be glycine. In this case the terminal glycine residue may be bonded to the hydrophobic portion through its N-terminus. If present, the protein transduction domain may be bonded to a cationic amino acid residue, e.g. arginine, lysine or histidine, through the C terminus of the cationic amino acid residue. The C terminus of the cationic amino acid residue may be bonded to the N-terminus of the protein transduction domain (e.g. to the Y of TAT).

The hydrophobic portion may be any suitable hydrophobic group such that the amphiphilic antimicrobial substance is capable of forming micelles in an aqueous matrix. The hydrophobic portion may be a C4 to C40 group (i.e. have from 4 to 40 carbon atoms), or C4 to C20, C4 to C10, C10 to C20, C20 to C30, C30 to C40, C15 to C25 or C25 to C35. It may have 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 carbon atoms. It may be a hydrocarbon group. It may be substituted. It may be unsubstituted. It may be linear. It may be branched. It may be cyclic. It may be bicyclic. It may be polycyclic. It may be aliphatic. It may have aromatic regions. It may be derived from a natural product. It may comprise, or may be, a steroid group, for example a cholesteryl group. It may be coupled to the oligopeptide portion by means of an amide linkage, or a carbamate (urethane) linkage, or some other suitable linkage. The hydrophobic portion may comprise, or may consist of, a hydrophobic polymer. The hydrophobic polymer may be biodegradable. It may for example be a polylactides, a poly(lactide-co-glycolide), a polycaprolactone, a polycarbonate or some other biodegradable polymer. The hydrophobic portion may be coupled to only one cationic oligopeptide portion, or it may be coupled to more than one cationic oligopeptide portion, e.g. 2, 3, 4, 5, 6, 7, 8, 9, 10 or more than 10 cationic oligopeptide portions. In the case where it is coupled to more than two cationic oligopeptide portions, the substance may be regarded as a star polymer or dendrimer. It may be in the form of core-shell structures in which the hydrophobic portion is in the core and the cationic oligopeptide portions are in the shell.

The antimicrobial substance may for example be CholG₃R₆TAT, or CholG₃K₆TAT, or CholG₃H₆TAT wherein Chol represents a cholesteryl group coupled to G by a urethane linkage and TAT represents YGRKKRRQRRR. In some instances, a mixture of antimicrobial substances, for example the three listed above, may be used.

The antimicrobial substance may be capable of forming micelles or nanoparticles. In particular it may be capable of forming micelles or nanoparticles in a polar (e.g. aqueous) matrix. The matrix is preferably a fluid matrix. The micelles or nanoparticles may be formed through a membrane dialysis method or by a solvent evaporation method or by an emulsion method. The micelles or nanoparticles may form spontaneously in the aqueous matrix. They may form without substantial mechanical action (e.g. without vigorous agitation, sonication etc.). As the substance is an amphiphile, having a hydrophobic portion at one end of the molecule and a hydrophilic (cationic oligopeptide) portion at the other end, it may be capable of self assembling in the appropriate matrix. In particular, in a polar matrix structures may be formed in which the hydrophobic portion is buried away from the polar matrix and the hydrophilic portion extends outwards from the hydrophobic portion towards the polar matrix. Suitable such structures are micelles or nanoparticles. These may be regarded as core-shell nanoparticles (or core-shell micelles) in which the core comprises the hydrophobic portion and the shell comprises the hydrophilic (cationic oligopeptide) portion. Therapeutical agents such as anticancer drugs or small molecular antibiotics may be incorporated into the core, for example through a membrane dialysis method or through a solvent evaporation method or through an emulsion method. The inventors consider that the cationic groups of the antimicrobial substance are related to its antimicrobial activity. Consequently a core-shell structure as described above would provide those cationic groups in the shell, enabling them to access and act upon microorganisms. Suitable polar matrices for inducing the self-assembly described above include aqueous matrices, e.g. water, saline solution, aqueous biological fluids (blood, saliva etc.) or other aqueous fluids. In the event that the shell comprises a protein transduction domain, this is likely to reside in the hydrophilic shell. This is likely to make the domain available to facilitate the micelles or nanoparticles in crossing the BBB and/or penetrating cells.

The micelles or nanoparticles of the antimicrobial substance commonly have a mean diameter of about 100 to about 700 nm, or about 100 to 500, 100 to 300, 300 to 500, 500 to 700 or 200 to 400 nm, e.g. about 100, 150, 200, 250, 300, 350, 400, 450, 500, 550, 600, 650 or 700 nm. They may be substantially monodispersed. This diameter will depend on the precise nature of the substance, including the size and structure of the hydrophobic portion and of the hydrophilic portion. They may have a low polydispersity index. The polydispersity index may be less than about 1, or less than about 0.5, 0.4 or 0.3, or may be about 0.1 to about 1, or about 0.25 to 1, 0.5 to 1, 0.1 to 0.5, 0.1, to 0.3 or 0.2 to 0.4, e.g. about 0.1, 0.15, 0.2, 0.25, 0.3, 0.35, 0.4, 0.45, 0.5, 0.55, 0.6, 0.65, 0.7, 0.75, 0.8, 0.85, 0.9, 0.95 or 1. They may have a zeta potential of greater than about 30, or greater than about 40, 50, 60, 70, 80 or 90 mV, or about 30 to 100, 40 to 100, 60 to 100, 80 to 100, 90 to 100, to 80, 30 to 60, 30 to 40, 60 to 80, 85 to 95 or 90 to 95 mV, e.g. about 30, 35, 40, 45, 50, 55, 60, 65, 70, 75, 80, 85, 90, 95 or 100 mV. They may be highly charged. They may have a high positive charge. Such charges provide substantial stability of the particles.

The antimicrobial substance may be active (optionally lethal) against a wide variety of microorganisms. It may be active against one or more of bacteria, yeast and fungus. It may be an antibacterial agent. It may be an anti-yeast agent. It may be an antifungal agent. It may be active against gram-positive bacteria. It may be active against other types of microorganism. It may have an minimum inhibitory concentration (MIC) against a target organism of less than about 20 micromolar, or less than about 15, 10 or 5 micromolar, or about 2 to about 20, about 5 to 20, 10 to 20, 2 to 10, 2 to 5 or 5 to 10 micromolar, e.g. about 2, 3, 4, 5, 6, 7, 8, 9, 10, 11, 12, 13, 14, 15, 16, 17, 18, 19 or 20 micromolar.

The antimicrobial substance may be capable of crossing the blood-brain barrier (BBB). It may be capable of crossing the BBB in sufficient quantity, or at a sufficient rate, so as to achieve a lethal dose towards the target microorganism in the brain of the subject. It may be capable of penetrating cell walls so as to enter cells.

The amphiphilic antimicrobial substance described herein may be made by a process comprising coupling a hydrophobic compound to a cationic oligopeptide.

The hydrophobic compound may correspond to the hydrophobic portion of the antimicrobial substance as described earlier. It may comprise the hydrophobic portion and a functional group coupled thereto (optionally directly bonded thereto), wherein the functional group is capable of reacting with an oligopeptide (for example with the N-terminus of an oligopeptide) so as to couple the hydrophobic portion to the oligopeptide (for example to the N-terminus of an oligopeptide). The functional group may be a haloformate ester (OC(═O)X, where X is a halogen), e.g. a chloroformate ester or a bromoformate ester, so as to form a carbamate linkage to the oligopeptide, or it may be an acid halide (C(═O)X, where X is a halogen), e.g. an acid chloride or acid bromide, so as to form an amide linkage to the oligopeptide, or it may be some other suitable functional group capable of reacting with the N-terminus of an oligopeptide so as to couple the hydrophobic portion to the oligopeptide.

In some embodiments, the spacer comprises a functional group. It may for example comprise amino acid residues bearing the functional group. In these embodiments the hydrophobic compound may be coupled to the cationic oligopeptide through that functional group. Thus if the functional group is a carboxylic acid, the hydrophobic compound may bear an amine or a hydroxyl, so as to couple to the cationic oligopeptide by means of an amide or ester group respectively. If the functional group is an amine group or a hydroxyl group, the hydrophobic compound may bear a carboxylic acid group, so as to couple by means of an amide or ester group respectively. Other suitable coupling reactions include “click” reactions. For example the cationic oligopeptide may be functionalised with an azide group and the hydrophobic group may contain an alkynyl group, whereby the two may be reacted to form a 1,2,3-triazole linkage.

The cationic oligopeptide may correspond to the cationic oligopeptide portion described earlier. It may comprise said oligopeptide portion having an NH₂ group as its N-terminus.

The step of coupling the hydrophobic compound to the cationic oligopeptide may comprise mixing a solution of the hydrophobic compound with a solution of the cationic oligopeptide. It may also comprise allowing sufficient time for the reaction to proceed. The sufficient time may be at least about 1 hour, or at least about 2, 3, 4, 6, 12, 18 or 24 hours, or may be about 1 to about 48 hours, or about 1 to 24, 1 to 12, 1 to 6, 6 to 48, 12 to 48, 24 to 48, 6 to 30, 12 to 30, 18 to 30 or 18 to 24 hours, e.g. about 1, 2, 3, 6, 12, 15, 18, 21, 24, 30, 36, 42 or 48 hours. The mixing and the subsequent reaction may, independently, be conducted at between about 0 and about 25° C., or about 0 to 20, 0 to 15, 0 to 10, 0 to 5, 5 to 25, 10 to 25 or 5 to 10° C., e.g. at about 0, 1, 2, 3, 4, 5, 10, 15, 20 or 25° C. The time for reaction may depend on the temperature used. Depending on the nature of the coupling reaction used, the coupling reaction may be base catalysed. Suitable bases include tertiary amines or pyridines, e.g. triethylamine, tripropylamine, pyridine etc. The solvent should be capable of dissolving both the hydrophobic compound and the cationic oligopeptide. In certain circumstances different solvents may be used for the cationic oligopeptide and for the hydrophobic compound. In this case, the different solvents may be miscible, and the solutions should be mixed in a ratio such that the resulting solvent mixture is capable of dissolving both the hydrophobic compound and the cationic oligopeptide. The hydrophobic compound may be used in a molar excess. It may be used in a molar excess of about 1.5 to about 20 (where molar excess is defined as the number of moles of hydrophobic compound used divided by the number of moles of cationic oligopeptide used), or about 2 to 20, 5 to 20, 10 to 20, 1.5 to 10, 1.5 to 5, 2 to 15, 5 to 15 or 5 to 10, e.g. about 1.5, 2, 3, 4, 5, 6, 7, 8, 9, 10, 11, 12, 13, 14, 15, 16, 17, 18, 19 or 20. Suitable solvents for use in the reaction include dipolar aprotic solvents such as dimethyl formamide, dimethyl sulfoxide, hexamethyl phosphoramide, dioxane, tetrahydrofuran etc. Following the reaction, the solvent may be removed. Commonly the residue will be washed with a suitable solvent capable of dissolving unreacted hydrophobic compound but not dissolving the antimicrobial compound product. Suitable solvents include diethyl ether. The product may then be further purified. Suitable methods include dialysis using a membrane with a molecular weight cutoff below the molecular weight of the product. Other suitable methods in certain cases may include preparative gel permeation chromatography and preparative hplc. In some cases combinations of such methods may be used.

The process may also comprise the step of making the cationic oligopeptide. This may be achieved by solid state synthesis or by other known methods.

In particular the cationic oligopeptide may be made by means of a peptide synthesiser. The method may use an Fmoc protecting group. Other suitable protecting groups include t-Boc. It may proceed from the C-terminus to the N-terminus of the oligopeptide. It may use a double coupling method. Thus in a typical amino acid addition step of the synthesis, an excess (e.g. about 5 mol equivalents) of amino acid together with an activator reagent and a molar excess of base (e.g. about 10 mol equivalents) are exposed to the resin (having the growing oligopeptide chain attached thereto). Suitable activator reagents include benzotriazol-1-yl-oxytripyrrolidinophosphonium hexafluorophosphate. Suitable bases include tertiary amines such as N-methylmorpholine. Removal of the Fmoc protecting group may be effected using mild base such as piperidine. The final formed oligopeptide may be separated from the resin using acid, such as trifluoroacetic acid, together with a suitable silane such as triisopropylsilane. The separated oligopeptide may be purified by suitable known methods such as hplc.

The process may additionally comprise the step of dispersing the antimicrobial substance in water so as to form nanoparticles or micelles of the antimicrobial substance in the water. A suitable means for achieving this comprises dissolving the antimicrobial substance in a water miscible solvent and dialysing the resulting solution against water using a dialysis membrane having a low molecular weight cut-off. Suitable solvents include dipolar aprotic solvents such as dimethyl formamide, dimethyl sulfoxide, dimethylacetamide, hexamethyl phosphoramide, dioxane, tetrahydrofuran etc. Preferably the water is purified water, e.g. deionised water, distilled water, reverse osmosis purified water or other suitably pure water. The cut-off of the dialysis membrane may be less than the molecular weight of the antimicrobial substance. It may for example be about 500 to about 1500, e.g. about 500, 1000 or 1500. The properties of the resulting micelles or nanoparticles have been described earlier.

The process may also comprise incorporating a hydrophobic substance into the cores of the nanoparticles or micelles. This may be accomplished by means of a membrane dialysis method or by means of a solvent evaporation method or by means of an emulsion method.

Thus the present invention also provides a micellar solution or a suspension of nanoparticles of the antimicrobial substance in an aqueous matrix. The nanoparticles, or micelles of the micellar solution, may comprise a hydrophobic substance. The hydrophobic substance may be located in hydrophobic cores of the nanoparticles or micelles. The hydrophobic substance may be a therapeutic substance. In this case the micellar solution or suspension may be useful for delivering the therapeutic substance. The aqueous matrix may be purified water, as described above, or it may be some other aqueous matrix. In this event, the micellar solution or suspension of nanoparticles may be elaborated by addition (optionally dissolution) of one or more other substances to the micellar solution or suspension. These substances may for example comprise salts for maintaining osmotic pressure, or may be adjuvants for the antimicrobial substance, or may be additional therapeutic agents to be used in conjunction with the antimicrobial substance, or may be some other type of substance. The quantity of such substances added will depend on their nature and required activity.

The antimicrobial substance may be used for killing microorganisms. Thus microorganisms exposed to the antimicrobial substance may be effectively killed. The antimicrobial substance may be in the form of an aqueous dispersion of nanoparticles or an aqueous micellar solution, as described earlier, or it may be used neat, in solution, as a cream or lotion or in some other suitable form, depending on the nature of the particular application. Thus it may be used for a live patient internally, systemically, topically, or may be used on a surface for disinfection.

In particular the antimicrobial substance may be used internally in a patient for treating an internal infection. In this case the substance may have low or negligible toxicity towards the patient. It may have sufficiently low toxicity that a dose of the antimicrobial substance that is effective to treat, control or cure the infection is non-toxic, or at least non-lethal, to the patient. It may show low toxicity at the MIC towards the target microorganism, or at the effective dose. The antimicrobial substance may be administered to the patient orally, or may be administered by injection (subdermally, intravenously, intramuscularly etc.) or it may be administered intranasally or it may be administered by some other route (e.g. by inhalation). In certain embodiments of the invention the antimicrobial substance is capable of passing across the BBB. This makes these embodiments particularly suited to treatment of infections in the brain of a patient.

The patient to which the antimicrobial substance is administered may be a human, or it may be a non-human animal. It may be a mammal, e.g. a non-human mammal. It may be a bird. It may be a fish. It may be a primate, e.g. a dog, a cat, a cow, a horse, a sheep, a goat, a mouse, a rat or some other primate. It may be a domestic animal. It may be a pet. It may be a farm animal. It may be a wild or undomesticated animal.

The antimicrobial substance may have low haemolytic activity. It may have low haemolytic activity against red blood cells. It may have shown less than about 30% haemolysis at the MIC towards the target microorganism, or less than about 20, 15, 10 or 5%, e.g. about 30, 25, 20, 25, 10, 9, 8, 7, 6, 5, 4, 3, 2, 1 or 0% haemolysis.

The amphiphilic antimicrobial substance may be biodegradable. It may be suitable for delivery of a hydrophobic therapeutic substance located in hydrophobic cores of micelles or nanoparticles of the antimicrobial substance.

### DETAILED DESCRIPTION OF THE PREFERRED EMBODIMENTS

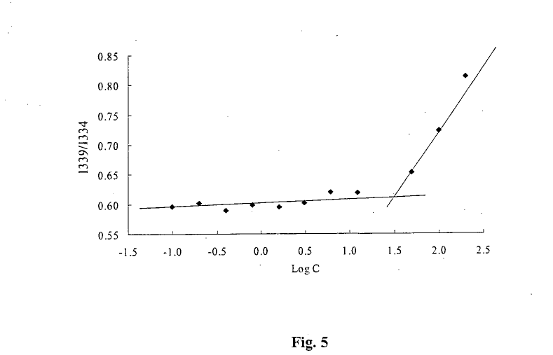

In one preferred embodiment the present invention relates to a cholesterol-grafted cationic peptide suitable for use as an antimicrobial agent of broad-spectrum activities for treatment of brain infections. This peptide contains a cholesterol moiety, three glycine residues as a spacer, six arginine residues and a cell-penetrating peptide, TAT. This peptide has a critical micelle concentration (CMC) of 31.6 mg/L (i.e. 10.1 μM) in de-ionized (DI) water (FIG. 5), and can easily self-assemble in aqueous solutions to form cationic core/shell structured nanoparticles at 31.6 mg/L or above. These nanoparticles are spherical and, have an average diameter of about 300 nm with zeta potential of 92 mV. They show low minimal inhibitory concentrations (MIC) of 10.7, 10.8 and 11.0 μM against Bacillus subtilis (bacterium), Candida albicans (yeast) and Stachybotrys chartarum (fungus) respectively, and display much stronger antimicrobial ability than cationic peptides without cholesterol. The inventors have observed that incubation with the nanoparticles induced pore formation on the surface of the yeast and rough surface of the bacterium. It also accelerates division of the bacterium, forming minicells. The interactions between the nanoparticles and cell wall lead to inhibition of cell-wall synthesis and thus osmotic lysis of cells. Importantly, it was demonstrated that the antimicrobial nanoparticles cross the blood-brain barrier (BBB) in a rat model. These cationic self-assembled peptide nanoparticles provide a promising antimicrobial agent against brain infection.

The invention is not limited to the particular preferred embodiment described herein. For example the lengths of the arginine residues or glycine residues of the peptides may be varied, and different hydrophobic groups may be used. In addition, arginine may be replaced with lysine (arginine in TAT: not included). In certain applications, TAT may not be present in the compounds.

The inventors describe herein cationic core/shell nanoparticles self-assembled from an amphiphilic peptide containing a cell-penetrating residue, and demonstrate that these nanoparticles possess strong antimicrobial activities. The low minimal inhibitory concentrations (MIC) of the nanoparticles are much lower than those of hydrophilic cationic peptides without the formation of nanoparticles. It was observed that incubation with the nanoparticles induced pore formation on the surface of the yeast, and rough surface as well as accelerated division of the bacterium, forming minicells.

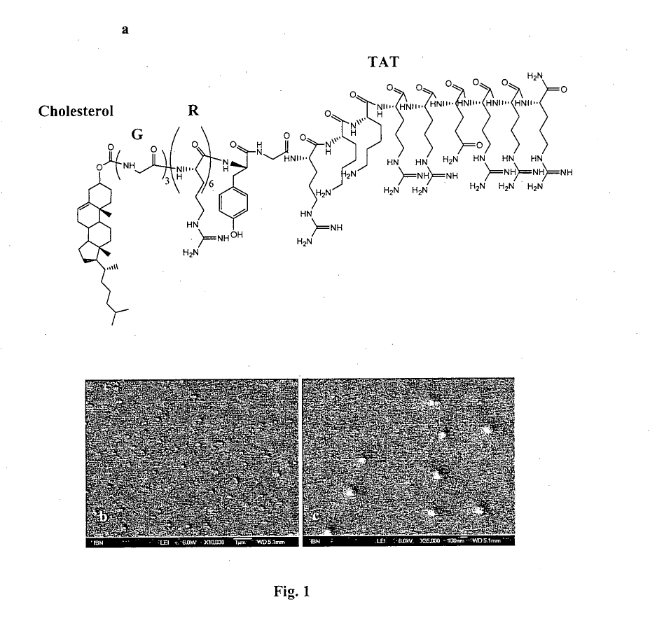

TAT (YGRKKRRQRRR) peptide is the protein transduction domain from the transcriptional activator Tat protein of the human immunodeficiency virus type-1 (HIV-1). After conjugation with TAT, proteins with molecular weight ranging from 36 to 119 kDa (Schwarze, S. R., Ho, A., Vocero-Akbani, A. & Dowdy, S. F. In vivo protein transduction: delivery of a biologically active protein into the mouse. Science 285, 1569-1572 (1999)) and quantum dots were able to cross the BBB (Santra, S., Yang, H., Stanley, J. T., Holloway, P. H., Moudgil, B. M., Walter, G. & Mericle, R. A. Rapid and effective labeling of brain tissue using TAT-conjugated CdS:Mn/ZnS quantum dots. Chem. Commun. 25, 3144-3146 (2005)). In the present work, an amphiphilic peptide (CholG₃R₆TAT) was constructed, containing the cell-penetrating peptide TAT, six arginine residues (R₆), three glycine moieties (G₃) as spacer and cholesterol (Chol) as the hydrophobic block (FIG. 1a). This peptide can easily form core/shell structured nanoparticles (i.e. micelles) having a hydrophobic cholesterol core and a hydrophilic cationic peptide shell with TAT molecules arranged towards the surrounding environment. The formation of nanoparticles is expected to increase local density of positive charges, enhancing antimicrobial properties of the cationic peptide. The presence of TAT molecules on the surfaces renders these nanoparticles capable of crossing the BBB for the treatment of brain infection.

G₃R₆TAT was synthesized by a solid-phase method. CholG₃R₆TAT was obtained by grafting cholesteryl chloroformate onto the N-terminus of G. This peptide can easily self-assemble in an aqueous solution to form nanoparticles. 10 mg of CholG₃R₆TAT was dissolved in 3 mL of dimethyl sulfoxide (DMSO), and dialyzed against 500 mL of de-ionized (DI) water at room temperature (22° C.) for 24 hours using a dialysis membrane with a molecular weight cut-off of 1,000 (Spectra/Por 7, Spectrum Laboratories Inc.). The external water phase was replaced every 6 hours. The resulting nanoparticles were characterized using a zeta potential analyzer with dynamic light scattering capability (ZetaPlus, Brookhaven, U.S.A.). Their effective diameter and zeta potential were 300 nm with polydispersity index of 0.25 and 92±2 mV respectively. The nanoparticles were spherical in nature and had a size less than 150 nm after self-drying under air (FIG. 1b).

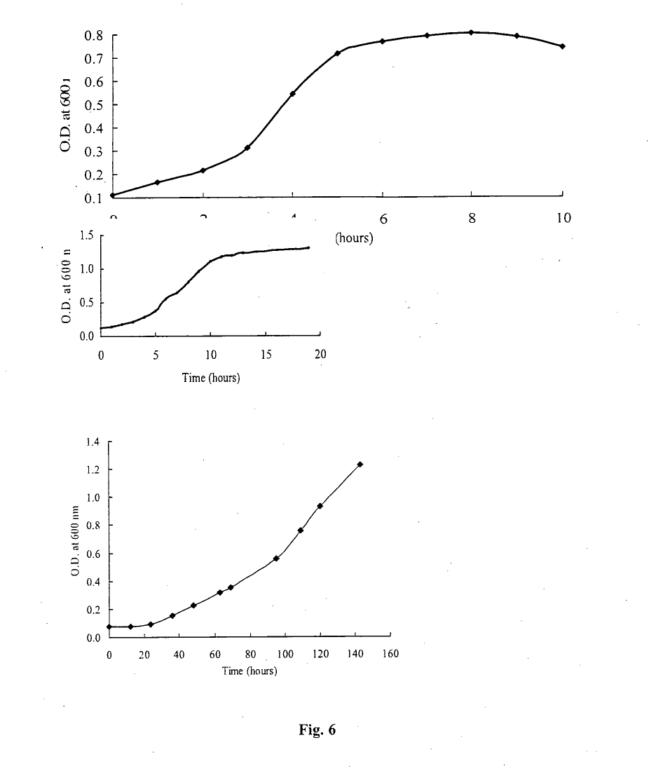

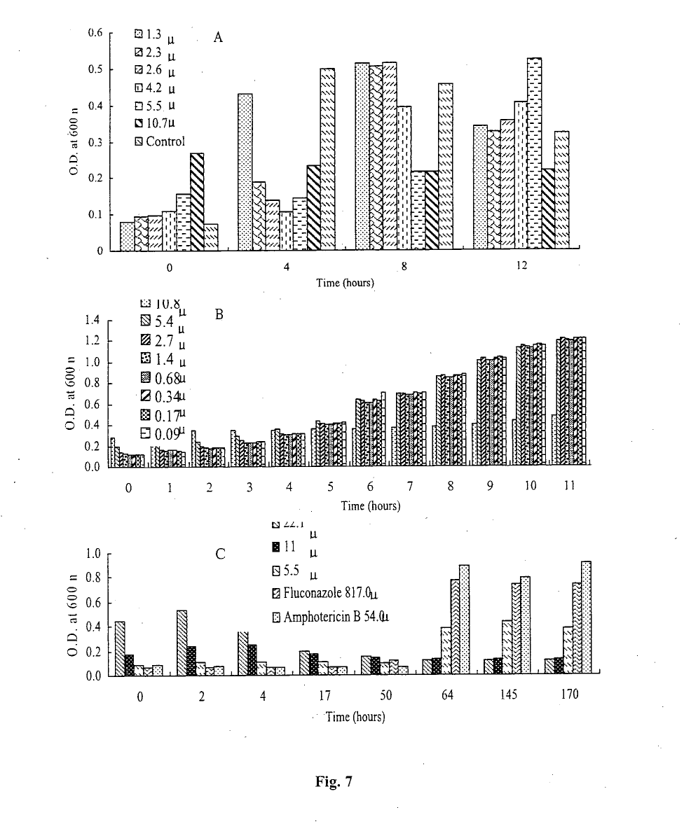

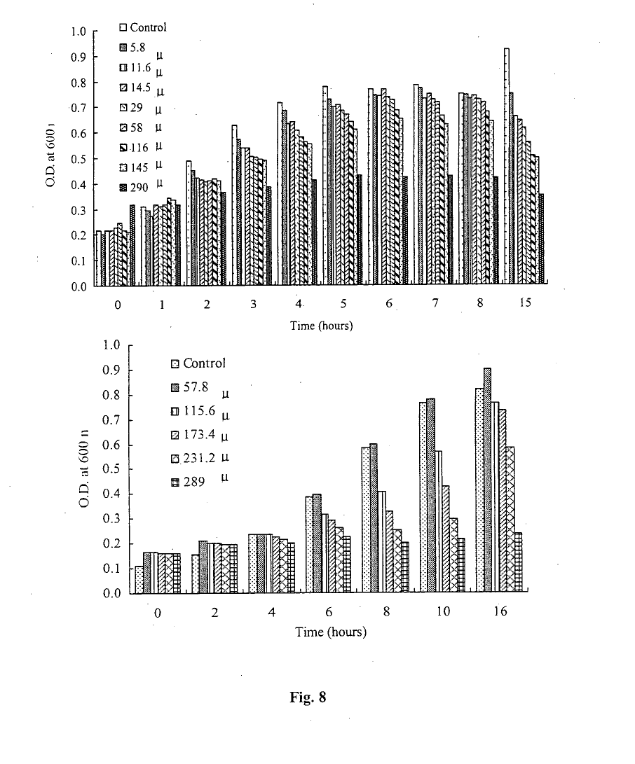

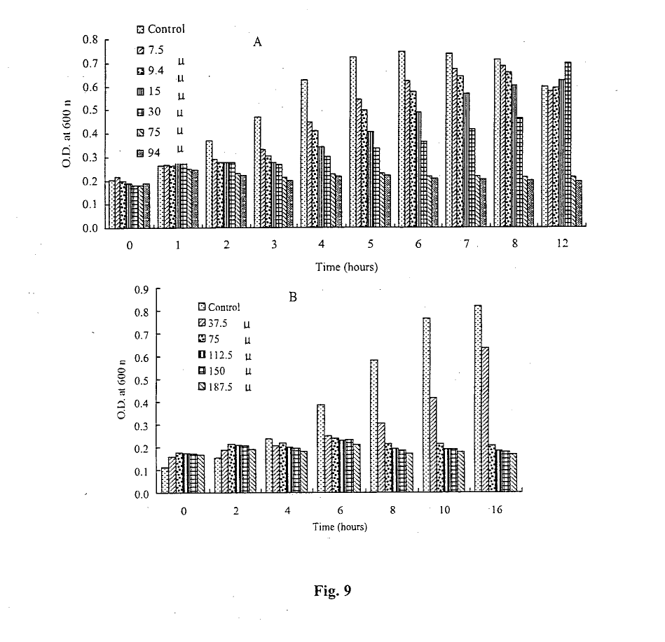

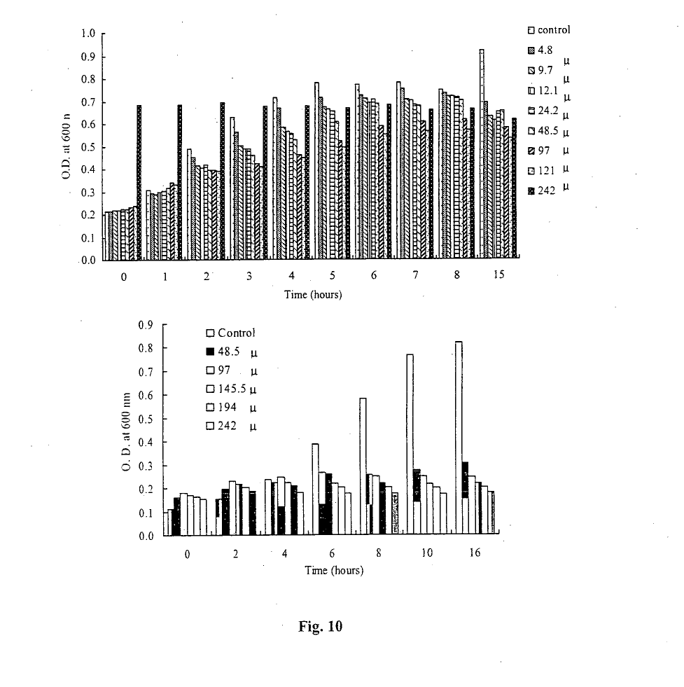

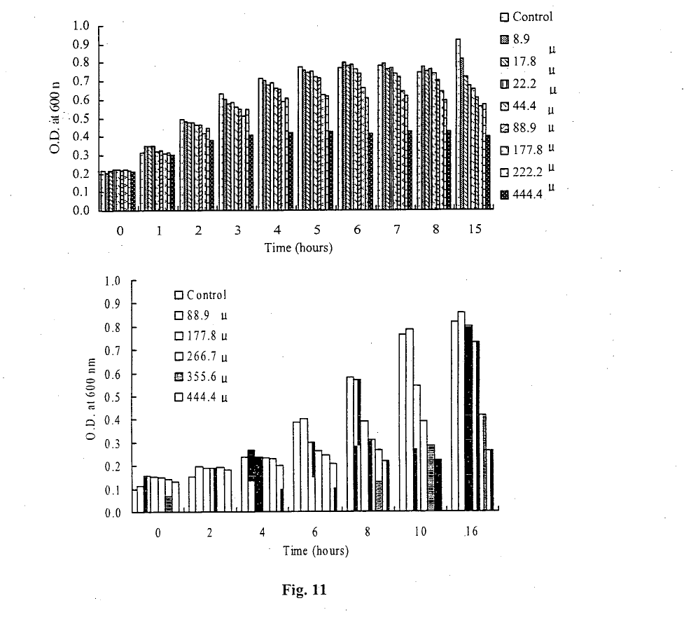

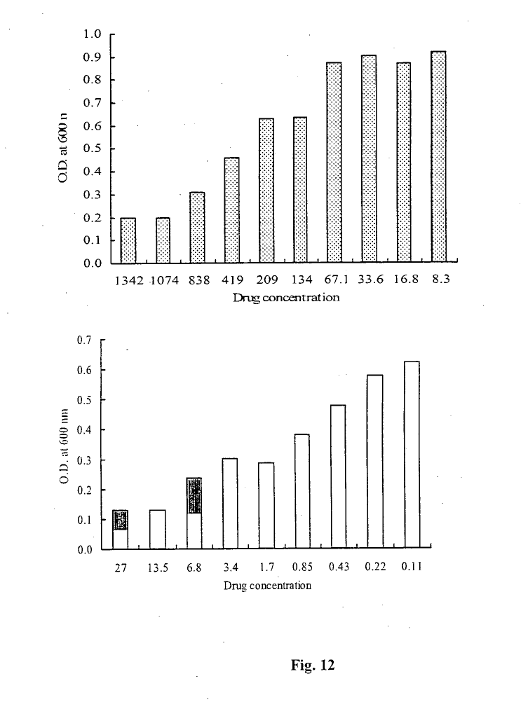

In clinical practice, meningitis patients are empirically treated with antibiotics of broad-spectrum antimicrobial activities prior to identifying specific pathogens as any delay in treatments may cause mortality and morbidity. Therefore, peptides as potential antimicrobial agents for combating brain infections must be able to kill both bacteria and fungi. The MICs of peptides and cationic peptide nanoparticles were evaluated against Bacillus subtilis (gram-positive bacterium), Candida albicans (yeast) and Stachybotrys chartarum (fungus). The nanoparticles exhibited both antibacterial and antifungal activities, and their MIC was 10.7, 10.8 and 11.0 μM against Bacillus subtilis, Candida albicans and Stachybotrys chartarum respectively (See FIGS. 6 and 7). G₃TAT had a low antimicrobial activity and its MIC against Bacillus subtilis and Candida albicans was 290.0 and 289.0 μM respectively (FIG. 8). Adding six arginine residues to TAT (i.e. G₃R₆TAT) significantly reduced MIC (290.0 and 289.0 vs. 75.0 μM for Bacillus subtilis and Candida albicans respectively) (FIG. 9). The presence of TAT did not merely provide positive charges since the MIC of G₃R₁₂ against Bacillus subtilis and Candida albicans was much higher than that of G₃R₆TAT (242.0 vs. 75.0 μM) (FIG. 10). Cell-penetrating property of TAT must play a role in inhibiting the growth of microbes, and the addition of TAT to G₃R₆ strongly enhanced its antimicrobial activity (MIC: 75.0 vs. >444.4 μM) (FIG. 11). However, the MIC of G₃R₆TAT was still much higher than that of the nanoparticles self-assembled from the amphiphilic peptide (10.7 vs. 75.0 μM). The formation of core/shell nanoparticles enhanced the antimicrobial ability of the peptide, resulting in lower MICs. In addition, the nanoparticles were much more powerful in inhibiting proliferation of Stachybotrys chartarum than the conventional antifungal agents such as fluconazole and amphotericin B (MIC: 11.0 μM vs. >817.0 and >54.0 μM respectively) (FIG. 7C). Moreover, the nanoparticles were also superior to the conventional antibiotics such as penicillin G and doxycycline in killing Bacillus subtilis (MIC: 11.0 vs. 6720 and 13.5 μM respectively) (FIG. 12).

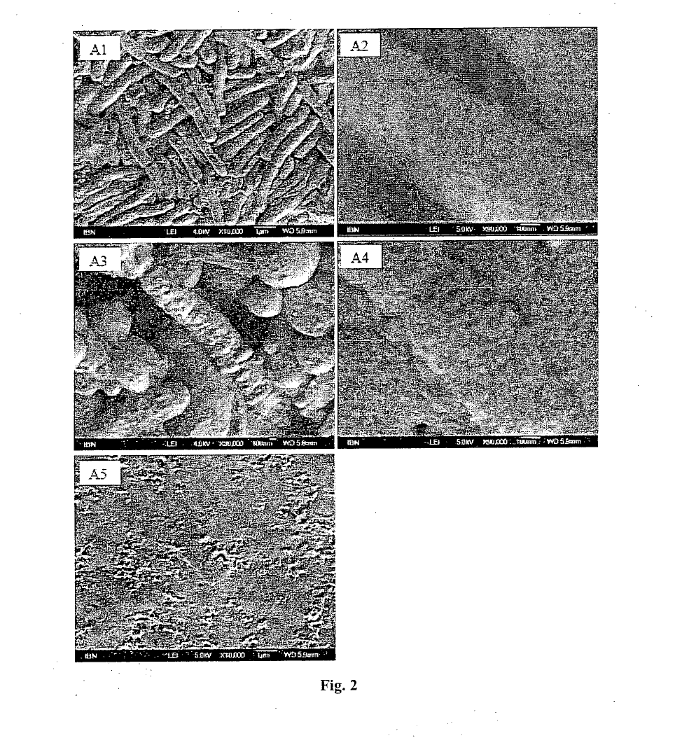

Next, morphological changes of Bacillus subtilis and Candida albicans were investigated before and after incubation with the nanoparticles at lethal doses for various periods of time. Untreated Bacillus subtilis exhibited smooth surface (FIGS. 2A1 and A2). In sharp contrast, the cell surface became extremely rough, and a large number of minicells were formed and cell debris was observed after treatment with the nanoparticles of 13.0 μM for 90 minutes (FIGS. 2A3 and A4). The treatment with the nanoparticles of 26.0 μM for 90 minutes led to more cell debris (FIG. 2A5). The formation of minicells was also observed in Bacillus subtilis treated with the cationic peptide antibiotic, nisin. The nanoparticles may have a similar mechanism of action as nisin against Bacillus subtilis. The uptake of the nanoparticles into the cell wall via non-specific electrostatic interaction accelerated cell division, causing the formation of minicells. The inventors consider that the steric hindrance that the nanoparticles provided in the cell wall and hydrogen bindings/electrostatic interaction between the cationic peptides and peptidoglycans of cell wall, which are made from polymers of alternating N-acetylglucosamine and N-acetylmuramic acid in β linkage, cross-linked by short peptide stems, might inhibit cell wall synthesis, leading to osmotic lysis of cells. Candida albicans underwent different morphological changes (FIG. 2B1 to B6). Numerous pores with a size less than 50 nm were formed on the cell surfaces after treatment of nanoparticles of 13.0 μM for 30 minutes (FIG. 2B3). Cell wall was efficiently disrupted and protoplasts were exposed after 100 minutes because of the inhibition of cell wall synthesis (FIGS. 2B4 and B5). At 200 minutes, the majority of protoplasts broke into debris due to osmotic lysis (FIG. 2B6). In addition to the osmotic lysis mechanism caused by the inhibition of cell-wall synthesis, the nanoparticles might permeate through the cytoplasmic membrane of both organisms due to the presence of TAT, destabilizing the membrane based on the electroporation and/or sinking raft model (Chan, D. I., Prenner, E. J. & Vogel, H. J. Tryptophan- and arginine-rich antimicrobial peptides: Structures and mechanisms of action. Biochimica et Biophysica Acta-Biomembranes 1758, 1184-1202 (2006)).

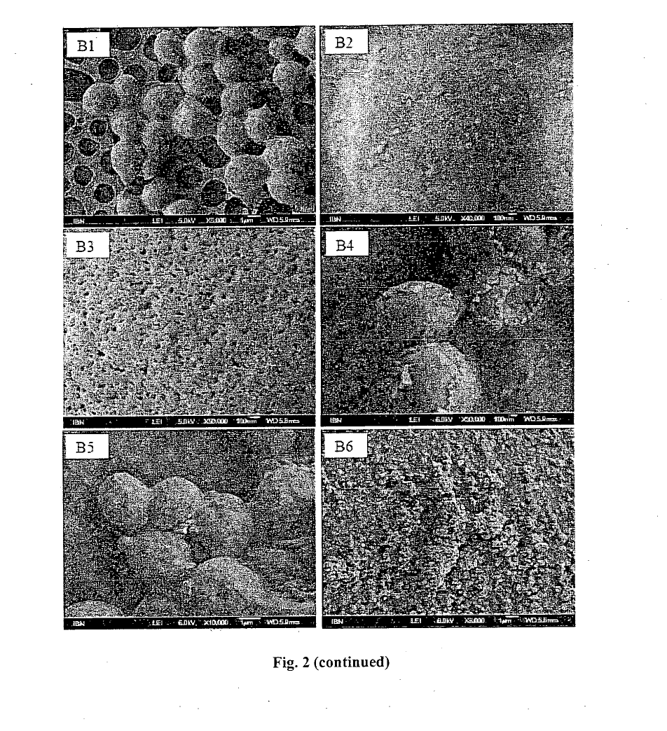

Further study was conducted regarding hemolysis induced after incubation of rat red blood cells with nanoparticles and amphotericin B. The nanoparticles showed low hemolytic activity at low concentrations (FIG. 3). At 16.0 μM (i.e. 50 mg/L), a concentration higher than the MIC, less than 20% hemolysis was observed with the nanoparticles, while amphotericin B mediated more than 90% hemolysis even at concentrations lower than its MIC.

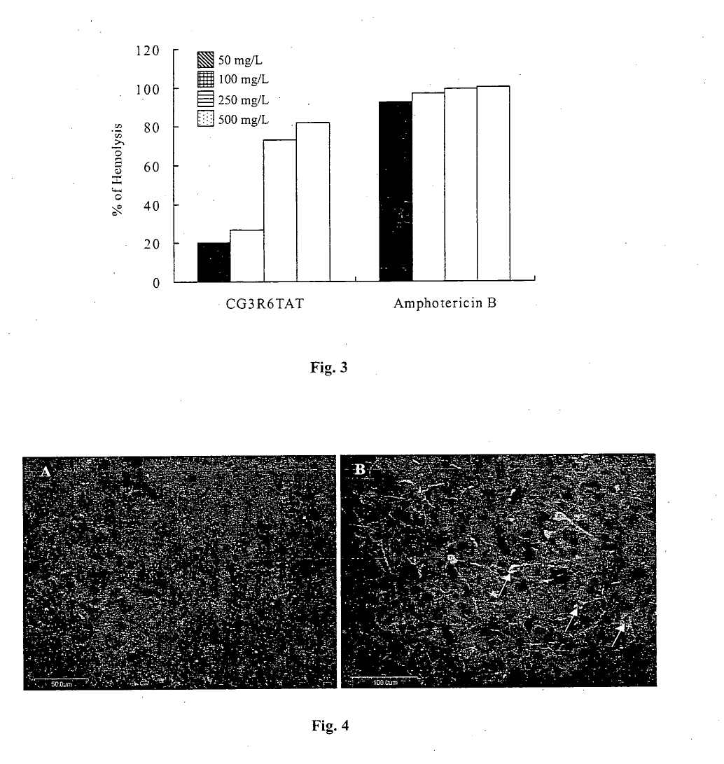

To determine whether the nanoparticles were able to cross the BBB, the distribution of FITC in hippocampus brain sections of rats was observed at 4 hours after i.v. injection of FITC or FITC-loaded nanoparticles. FITC was first loaded into CholG₃R₆TAT nanoparticles. 0.35 mg of FITC and 2.3 mg of CholG₃R₆TAT were dissolved in 1 mL of DMSO, which was dialyzed against 500 mL of DI water for three days at 10° C. using a dialysis bag with a molecular weight cut-off of 1,000 Da. The external water phase was replaced six times. FITC content was 5.3% in weight and the effective diameter of FITC-loaded nanoparticles was 356 nm. Naked FITC was unable to cross the BBB (FIG. 4A). In contrast, FITC-loaded nanoparticles crossed the BBB, principally surrounding the nuclei of neurons (FIG. 4B, white arrows).

In conclusion, it has been demonstrated that the amphiphilic peptide CholG₃R₆TAT is able to self-assemble into cationic core/shell nanoparticles. These nanoparticles possessed a broad spectrum of antimicrobial activities. They are efficient in inhibiting growth of both bacteria (gram-positive) and fungi with low MIC yet induce relatively low hemolysis. In addition, they are able to cross the BBB, providing a great potential in treating brain infections.

### Example

### Peptide Synthesis

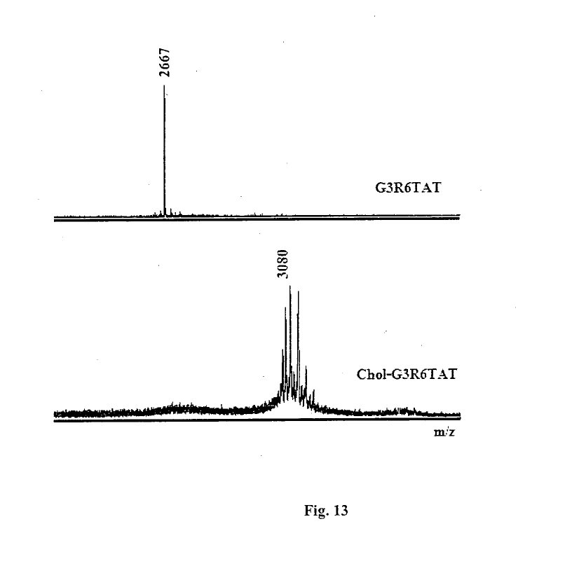

GGGRRRRRRYGRKKRRQRRR (G₃R₆TAT) was synthesized according to the 9-fluorenylmethoxycarbonyl (Fmoc) approach using an Apex 396 peptide synthesizer (Aapptec, U.S.A.). The peptide was assembled on Fmoc-Arg(Pbf)-Rink Amide-MBHA resin (LC Sciences, U.S.A.) at 0.1 mmol scale using a double coupling method. Briefly, resin was reacted with 5 equivalents of amino acids, 5 equivalents of activator reagent, benzotriazol-1-yl-oxytripyrrodinophosphonium hexafluorophosphate (PyBOP, LC Sciences, U.S.A.) and 10 equivalents of base, N-methylmorpholine (NMM, Merk). The Fmoc group was removed by gentle agitation in 20% of piperidine (Merk) in dimethylformamide (DMF, Sigma-Aldrich). After peptide synthesis, cleavage of the peptides from the resin was carried out with a mixture of trifluoroacetic acid (TFA, Merk), triisopropylsilane (TIS, Merk) and water in a volume ratio of 95:2.5:2.5 for 4-6 hours. The solution was concentrated by rotary evaporation, followed by precipitation in cold diethyl ether (Sigma-Aldrich). The crude peptide was collected by filtration and dried under vacuum. The crude peptide was further purified using high performance liquid chromatography (HPLC) consisting of a Waters 2767 sample manager, a Waters 996 PDA detector (Waters Corporation, U.S.A.) and a Grace Vydac C₁₈ column (10×250 mm). The mobile phase was composed of water containing 0.1% TFA and acetonitrile containing 0.1% TFA, and the volume percentage of acetonitrile was gradually increased from 5% to 40% in 20 minutes at a flow rate of 8 mL/min. The peptide was characterized by analytical reverse phase HPLC and matrix-assisted laser desorption ionization of time-of-flight (MALDI-TOF) mass spectrometry (Autoflex II, Bruker Daltronics) (FIG. 13). The purity of peptide was found to be about 95% according to HPLC analysis.

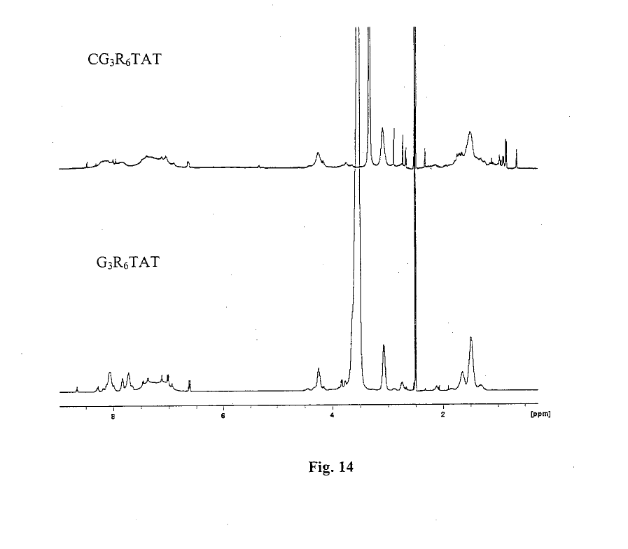

CholG₃R₆TAT was obtained by grafting cholesteryl chloroformate onto G₃R₆TAT via the N-terminus of G. Cholesteryl chloroformate (Sigma-Aldrich, 148 mg) dissolved in 15 mL of DMF was slowly added to 5 mL of DMF containing 70 μL of triethylamine (Fluka) and 88 mg of G₃R₆TAT at 0° C. with stirring. After 24 hours of reaction, DMF was removed from the mixture by purging dry nitrogen gas, and the mixture was then rinsed with diethyl ether for three times to remove unreacted cholesteryl chloroformate. The crude product was further purified by dialysis against DMF for six days using a membrane with a molecular weight cut-off of 1,000 Da. DMF was then removed by vacuum drying to yield a final product. The successful synthesis of CholG₃R₆TAT was evidenced by MALDI-TOF and ¹H-NMR analyses (See FIGS. 13 and 14).

### Minimal Inhibitory Concentration (MIC) Determination.

Bacillus subtilis, Candida albicans and Stachybotrys chartarum (ATCC) were grown in tryptic soy broth at 37° C., yeast mold broth at 24° C. and tryptic soy broth at 26° C., respectively. The MICs of the peptides or peptide nanoparticles were measured using a broth microdilution method. Briefly, 50 μL of peptide and peptide nanoparticle solutions with a concentration of 7.1 to 142 μM was placed into each well of 96-well plates. 50 μL of microorganism solution was added to each well to give an optical density reading of 0.1 to 0.2 at 600 nm. The cell cultures were then incubated for 15, 12/16 and 170 hours for Bacillus subtilis, Candida albicans and Stachybotrys chartarum respectively, and the MIC was taken at the concentration at which no growth was observed. Broth containing cells alone was used as control. The tests were repeated three times.

### Scanning Electron Microscopy (SEM).

The morphologies of the peptide nanoparticles and microorganisms before and after treatment with peptides or peptide nanoparticles were observed using a field emission SEM (JEOL JSM-7400F) operated at an accelerating voltage of 5.0 keV. For peptide nanoparticles, 20 μL of the nanoparticle solution was placed on a silicon wafer, and air-dried at room temperature. The wafer was mounted on aluminum stud, and then coated with platinum for visualization.

The microorganisms grown in broth alone or incubated with peptides or peptide nanoparticles were harvested by centrifugation at 2500 g for 10 minutes. Cells were washed with phosphate-buffered saline (PBS) for three times and then fixed in PBS containing 5% formaldehyde for one day. The cells were further washed with DI water before being dehydrated using a series of ethanol washes and dried in a critical point dryer (Autosamdri-815, Tousimis Research Corporation, U.S.A.) and mounted onto aluminum stubs. The samples were coated with platinum prior to SEM analyses.

### Hemolysis Assays.

Fresh rat red blood cells were washed with PBS for three times. 100 μL of red blood cells suspended in PBS (4% in volume) was placed in each well of 96-well plates and 100 μL of peptide nanoparticle or amphotericin B solution was added to each well. The plates were incubated for one hour at 37° C. The cell suspensions were taken out and centrifuged at 1000 g for 5 minutes. Aliquots (100 μL) of supernatant were transferred to 96-well plates, and hemoglobin release was monitored at 576 nm using a microplate reader (Bio-Teck Instruments, Inc). Percentage of hemolysis was calculated using the following formula: Hemolysis (%)=[(O.D._{576 nm} in the nanoparticle solution−O.D._{576 nm} in PBS)/(O.D._{576 nm} in 0.1% Triton X-100−O.D._{576 nm} in PBS)]×100. In vivo studies.

All procedures involving animals were approved by the DSO IACUC committee and performed according to the guidelines set forth in the National Institutes of Health Guide for the Care and Use of Laboratory Animals (NIH Publications NO. 85-23, revised 1996). SD adult rats (250 g in weight) of 10 weeks old were injected with pure FITC or FITC-loaded nanoparticle solution via tail vein. Animals were sacrificed at 4 h post-injection. They were perfused with Ringer's solution, followed by 4% paraformaldehyde (pH 7.4). Following the perfusion, the brains were removed and kept in a similar fixative for 2 h. They were then kept in 0.1M phosphate buffer containing 20% sucrose overnight at 4° C. Frozen coronal sections of the cerebrum of 30 μm thickness were cut and rinsed in PBS with a cryostat and mounted on slides. The specimens were observed with a confocal microscope (Olympus Fluoview TM1000).

1. An amphiphilic antimicrobial substance comprising a hydrophobic portion coupled to a cationic oligopeptide portion.

2. The antimicrobial substance of claim 1 wherein the cationic oligopeptide portion comprises arginine residues and/or lysine residues.

3. The antimicrobial substance of claim 1 or claim 2 wherein the cationic oligopeptide portion is between 5 and 35 peptide units in length.

4. The antimicrobial substance of any one of claims 1 to 3 wherein the cationic oligopeptide portion comprises a protein transduction domain.

5. The antimicrobial substance of claim 4 wherein the protein transduction domain is a terminal domain.

6. The antimicrobial substance of claim 4 or claim 5 wherein the protein transduction domain is TAT (YGRKKRRQRRR).

7. The antimicrobial substance of any one of claims 4 to 6 wherein the protein transduction domain is coupled to a cationic oligopeptide group.

8. The antimicrobial substance of claim 7 wherein the cationic oligopeptide group comprises arginine groups and/or lysine groups.

9. The antimicrobial substance of claim 8 wherein the cationic oligopeptide group has from 2 to about 15 lysine and/or arginine groups.

10. The antimicrobial substance of any one of claims 7 to 9 wherein the cationic oligopeptide group is coupled to the hydrophobic portion by means of a linker group.

11. The antimicrobial substance of claim 10 wherein the linker group is an oligopeptide group.

12. The antimicrobial substance of any one of claims 7 to 11 wherein the cationic oligopeptide group is R₆ and the linker group is G₃, wherein the terminal glycine residue is bonded to the hydrophobic portion through its N-terminus.

13. The antimicrobial substance of any one of claims 1 to 12 wherein the hydrophobic portion is a C4 to C40 group or a hydrophobic biodegradable polymer.

14. The antimicrobial substance of any one of claims 1 to 13 wherein the hydrophobic portion comprises a steroid group.

15. The antimicrobial substance of claim 14 wherein the steroid group is a cholesteryl group.

16. The antimicrobial substance of any one of claims 1 to 15 which is CholG₃R₆TAT, wherein Chol represents a cholesteryl group and TAT represents YGRKKRRQRRR.

17. The antimicrobial substance of any one of claims 1 to 16, said substance being in the form of micelles or nanoparticles.

18. The antimicrobial substance of claim 17 wherein the micelles or nanoparticles have a mean diameter of about 100 to about 700 nm.

19. The antimicrobial substance of any one of claims 1 to 18, said substance having a MIC against each of Bacillus subtilis, Candida albicans and Stachybotrys chartarum of less than about 15 micromolar.

20. A process for making an amphiphilic antimicrobial substance according to any one of claims 1 to 19, said process comprising coupling a hydrophobic compound to a cationic oligopeptide.

21. The process of claim 20 wherein said coupling comprises reacting the hydrophobic compound with the N-terminus of the cationic oligopeptide or with a functional group in the cationic oligopeptide.

22. The process of claim 21 wherein the cationic oligopeptide comprises an uncharged oligopeptide spacer having a cationic oligopeptide group coupled to its C-terminus, said cationic oligopeptide group having a protein transduction domain coupled to its C-terminus.

23. The process of any one of claims 20 to 22 wherein the hydrophobic compound is a haloformate ester.

24. The process of any one of claims 20 to 23 additionally comprising the step of dispersing the antimicrobial substance in water so as to form nanoparticles or micelles of the antimicrobial substance in the water.

25. The process of claim 24 wherein the nanoparticles or micelles each comprise a hydrophobic core surrounded by a hydrophilic shell and the process comprises incorporating one or more therapeutic agents into the core of the nanoparticles or micelles.

26. A method for killing microorganisms comprising exposing said microorganisms to an antimicrobial substance according to any one of claims 1 to 19.

27. The method of claim 26 wherein the microorganisms are selected from the group consisting of bacteria, yeast and fungus and mixtures of any two or all of these.

28. The method of claim 26 or claim 27 wherein the concentration of the antimicrobial substance to which the microorganisms is exposed is less than about 15 micromolar.

29. The method of any one of claims 26 to 28 wherein the microorganisms are pathogens located in a patient and the step of exposing comprises administering said antimicrobial substance to the patient.

30. The method of claim 29 wherein the pathogens are located in the brain of the patient and the step of exposing comprises allowing the antimicrobial substance to cross the blood-brain barrier of said patient.

31. Use of an antimicrobial substance according to any one of claims 1 to 19 for the manufacture of a medicament for the treatment of an infection in a subject, said antimicrobial substance being effective in treatment of said infection.

32. Use according to claim 31 wherein said infection is an infection of the brain of the subject.

33. Use of an antimicrobial substance according to any one of claims 1 to 19 in therapy.

34. A pharmaceutical composition comprising an antimicrobial compound according to any one of claims 1 to 19 together with one or more pharmaceutically acceptable carriers, diluents and/or adjuvants.

35. The pharmaceutical composition of claim 34 wherein the antimicrobial compound is in the form of nanoparticles or micelles in an aqueous matrix.

36. The pharmaceutical composition of claim 35 wherein the nanoparticles or micelles each comprise a hydrophobic core surrounded by a hydrophilic shell and one or more therapeutic agents are present in the cores of the nanoparticles or micelles.
